(19) United States
(12) Patent Application Publication
(10) Pub. No.: US 2010/0106000 A1
(43) Pub. Date: Apr. 29, 2010

(54) Methods and Systems for Evaluating Glycemic Control

(75) Inventor: Andreas Thomas, Pirna (DE)

Correspondence Address: MEDTRONIC MINIMED INC., 18000 DEVONSHIRE STREET, NORTHRIDGE, CA, 91325-1219, US

(73) Assignee: MEDTRONIC MINIMED, INC., Northridge, CA (US)

(21) Appl. No.: 12/604,685

(22) Filed: Oct. 23, 2009

Related U.S. Application Data
(60) Provisional application No. 61/108,753, filed on Oct. 27, 2008.

(51) Int. Cl. A61B 5/145; A61M 5/14

(52) U.S. Cl. 600/365; 604/890.1

(57) ABSTRACT

A method of evaluating glycemic control of a patient includes providing a pentagon having five axes radiating from a center of the pentagon. A first pentagon area formed by a first point, a second point, a third point, a fourth point, and a fifth point plotted on the five axes, respectively, is determined. A second pentagon area formed by a sixth point, a seventh point, an eighth point, a ninth point, and a tenth point plotted on the five axes, respectively, is determined. A glycemic control parameter is determined based on the first pentagon area and the second pentagon area.

### FIELD OF THE INVENTION

Embodiments of the present invention are directed to systems and methods for evaluating glycemic control of a patient. Specifically, embodiments of the present invention are directed to providing an integrated description of glycemia in diabetic patients over a specific time interval, while including independent factors for assessing metabolic control.

### BACKGROUND OF THE INVENTION

Self-monitoring blood glucose and measuring glycated hemoglobin (HbA_{1c} or hemoglobin A_{1c}) values have become the established methods of assessing glycemic control of patients with diabetes. For these patients, who typically receive insulin treatments, the primary benefits of monitoring blood glucose levels 4 to 6 times a day are the ability to adapt their medication therapy themselves to their food intake and levels of physical activity, and to correct for non-physiological glycemic excursions. Therapists are particularly interested in the HbA_{1c} value, because it helps them assess metabolic quality. Besides being easy to measure, this parameter is especially valuable because of the established correlation between protein glycosylation and the development of diabetic complications—a relationship that has been demonstrated in large clinical studies. This correlation has allowed the HbA_{1c} value to gain acceptance as a target parameter in numerous national and international diabetes treatment guidelines. The simplicity in measuring and ease for a physician to interpret the HbA_{1c} value has made it the standard parameter for the evaluation of glycemic control.

At the same time, the HbA_{1c} value has been shown to correlate well with the mean glycemic level over the course of 8 to 12 weeks. This parameter, however, only represents part of the risk of disruptions in glucose homeostasis, namely the long-term profile; it does not describe acute fluctuations in blood and tissue glucose levels (i.e., glycemic variability) because glycosylated hemoglobin is present only in its labile aldimine state for the first six hours after formation. The stable ketoamine form only arises afterwards.

The significance of these glycemic variations is particularly clear with regard to the correlation between postprandial hyperglycemia and cardiovascular disorders, which was demonstrated as early as the 1990s in several studies. A further study performed during this time period shows that increased postprandial glucose excursions are associated with microvascular complications in patients with Type 2 diabetes. In-vitro studies performed on cells in which fluctuating glucose levels produce the greatest degree of oxidative stress, along with the highest rate of apoptosis, underscore the significance of glycemic variability. There are also indications that non-physiologically high postprandial excursions in patients with Type 2 diabetes are at the center of a cascade of diabetogenic and atherogenic events, such as increased insulin resistance, postprandial dyslipidemia, increased oxidative stress, a shift in the equilibrium in the coagulation cascade, endothelial dysfunction, etc. This problem is also relevant to patients with Type 1 diabetes. A Finnish study conducted over the course of 18 years was able to demonstrate that there are no significant differences between patients with Type 1 and Type 2 diabetes with respect to cardiovascular and overall mortality. To qualify these findings, however, it should be noted that no suitable prospective, randomized end-point studies have been conducted to this point that prove a clear association between glycemic variations and microvascular/macrovascular events.

Hypoglycemic excursions, on the other hand, also contribute to an increase in glycemic variability. Associated with this is an adrenergic reaction that, at least in patients with existing vascular damage, increases the risk of severe complications, such as myocardial infarction and apoplectic stroke.

Continuous glucose monitoring (CGM) makes it possible to characterize a patient's glycemic profile in detail over the course of at least a few days. CGM systems have been available on the market since 1999 and are becoming increasingly accepted for diabetological diagnostics. CGM software works up data from recorded glucose profiles and calculates a variety of different parameters for glucose profile characterization; standardization is not yet a possibility, however. The following parameters for describing glycemic control have been suggested in the literature (in some cases in combination with each other):
mean glucose concentration
standard deviation for the mean glucose concentration
the mean amplitude of glycemic excursions (MAGE), which describes the arithmetic mean of the difference between consecutive glycemic maxima and minima
the number of hypoglycemic and hyperglycemic events
the portion of each day spent in the hypoglycemic or hyperglycemic range
the percentage of time spent each day in the euglycemic range
mean of the maximum excursions in the hypoglycemic or hyperglycemic range
CONGA (continuous overall net glycemic action)
glucose lability index (LI)
average daily risk range (ADRR), which encompasses both the low and high blood glucose indices (LBGI and HBGI)
GRADE (glycemic risk assessment diabetes equation).

The essential difference between these parameters lies in the treatment of hypoglycemic excursions. MAGE and the standard deviation of the mean glucose level only take these into consideration indirectly, for instance, whereas ADRR and GRADE treat them directly. With the exceptions of MAGE and mean glucose concentration (indirectly via the relationship to HbA_{1c}), these various parameters have not been evaluated with respect to the quality of metabolic control and the risk of developing complications of diabetes. It follows that no verifiable conclusions may be drawn at the present time regarding the relationship between parameters such as these, which describe acute glycemia, and the HbA_{1c} value, which describes long-term metabolic control.

Despite the availability of analysis software, a detailed assessment of glucose profiles would be somewhat time consuming. This reason, along with other reasons (such as cost), constitutes an important reason why practical application of CGM has been relatively infrequent to date. In other words, it is difficult to obtain a quick overview from these measurements and to reach conclusions for the prognosis of diabetic complications. As such, quickly filtering core parameters from recorded glucose profiles and making them available in such a way that they may be applied and interpreted at a glance for a rapid assessment of a patient's glycemic profile would be an extremely worthwhile project.

It would be advantageous to have a simple, straightforward model based on various parameters that are either available from glucose profiles or that may be calculated quickly from profiles. It is desirable to create a model that yields a value that characterizes the course of acute and long-term glycemia.

### SUMMARY OF THE INVENTION

A method of evaluating glycemic control of a patient includes providing a pentagon having five axes radiating from a center of the pentagon. A first axis has a length representing a range of hemoglobin A_{1c} values. A second axis has a length representing a range of standard deviation of glucose values. A third axis has a length representing a range of amount of time per day values exceeding a first limit. A fourth axis has a length representing a range of daily area-under-curve values exceeding a second limit. A fifth axis has a length representing a range of mean glucose values. A first point on the first axis indicative of a representative hemoglobin A_{1c} value is plotted. A second point on the second axis indicative of a representative standard deviation of glucose value is plotted. A third point on the third axis indicative of a representative amount of time per day value exceeding the first limit is plotted. A fourth point on the fourth axis indicative of a representative daily area-under-curve value exceeding a second limit is plotted. A fifth point on the fifth axis indicative of a representative mean glucose value is plotted. A sixth point on the first axis indicative of a hemoglobin A_{1c} value of the patient is plotted. A seventh point on the second axis indicative of a standard deviation of glucose value of the patient is plotted. An eight point on the third axis indicative of an amount of time per day value exceeding the first limit of the patient is plotted. A ninth point on the fourth axis indicative of a daily area-under-curve value exceeding the second limit of the patient is plotted. A tenth point on the fifth axis indicative of a mean glucose value of the patient is plotted. A first pentagon area formed by the first point, the second point, the third point, the fourth point, and the fifth point is determined. A second pentagon area formed by the sixth point, the seventh point, the eighth point, the ninth point, and the tenth point is determined. A glycemic control parameter is determined based on the first pentagon area and the second pentagon area.

The glycemic control parameter may be determined by dividing the second pentagon area by the first pentagon area. The representative hemoglobin A_{1c} value, the representative standard deviation of glucose value, the representative amount of time per day value exceeding the first limit, the representative daily area-under-curve value exceeding the second limit, and the representative mean glucose value may be representative of a non-diabetic individual. The first limit may be 160 mg/dL. The second limit may be 160 mg/dL. The method may be implemented on a computing device. The method may be implemented on an infusion device. The method may be implemented on an infusion device controller/programmer. The method may be implemented on a medical device. The first axis representing the range of hemoglobin A_{1c} values and the fifth axis representing the range of mean glucose values may be adjacent to each other in the pentagon.

An article of manufacture containing code for evaluating glycemic control of a patient, comprising a computer-usable medium including at least one embedded computer program that is capable of causing at least one computer to perform providing a pentagon having five axes radiating from a center of the pentagon. A first axis has a length representing a range of hemoglobin A_{1c} values. A second axis has a length representing a range of standard deviation of glucose values. A third axis has a length representing a range of amount of time per day values exceeding a first limit. A fourth axis has a length representing a range of daily area-under-curve values exceeding a second limit. A fifth axis has a length representing a range of mean glucose values. A first point on the first axis indicative of a representative hemoglobin A_{1c} value is plotted. A second point on the second axis indicative of a representative standard deviation of glucose value is plotted. A third point on the third axis indicative of a representative amount of time per day value exceeding the first limit is plotted. A fourth point on the fourth axis indicative of a representative daily area-under-curve value exceeding a second limit is plotted. A fifth point on the fifth axis indicative of a representative mean glucose value is plotted. A sixth point on the first axis indicative of a hemoglobin A_{1c} value of the patient is plotted. A seventh point on the second axis indicative of a standard deviation of glucose value of the patient is plotted. An eight point on the third axis indicative of an amount of time per day value exceeding the first limit of the patient is plotted. A ninth point on the fourth axis indicative of a daily area-under-curve value exceeding the second limit of the patient is plotted. A tenth point on the fifth axis indicative of a mean glucose value of the patient is plotted. A first pentagon area formed by the first point, the second point, the third point, the fourth point, and the fifth point is determined. A second pentagon area formed by the sixth point, the seventh point, the eighth point, the ninth point, and the tenth point is determined. A glycemic control parameter is determined based on the first pentagon area and the second pentagon area.

The glycemic control parameter may be determined by dividing the second pentagon area by the first pentagon area. The representative hemoglobin A_{1c} value, the representative standard deviation of glucose value, the representative amount of time per day value exceeding the first limit, the representative daily area-under-curve value exceeding the second limit, and the representative mean glucose value may be representative of a non-diabetic individual. The first limit may be 160 mg/dL. The second limit may be 160 mg/dL. The article may be a computing device. The article may be an infusion device. The article may be an infusion device controller/programmer. The article may be a medical device. The first axis representing the range of hemoglobin A_{1c} values and the fifth axis representing the range of mean glucose values may be adjacent to each other in the pentagon.

### BRIEF DESCRIPTION OF THE DRAWINGS

FIG. 1 illustrates a Glucose Pentagon according to embodiments of the present invention.

FIG. 2 illustrates a Glucose Pentagon comparing a pentagon of a non-diabetic person to a pentagon of a diabetic patient according to embodiments of the present invention.

FIG. 3 illustrates a representative relationship between a Glycemic Risk Parameter (GRP) value and the risk of developing diabetic complications according to embodiments of the present invention.

FIGS. 4A-4C illustrate sample continuous glucose monitoring (CGM) profiles over three days and the resultant Glucose Pentagons for a representative diabetic patient according to embodiments of the present invention.

FIGS. 5A-5D illustrate Glucose Pentagons for a selected day for a plurality of diabetic patients.

FIG. 6 illustrates a flow chart of evaluating glycemic control of a patient according to embodiments of the present invention.

### DETAILED DESCRIPTION

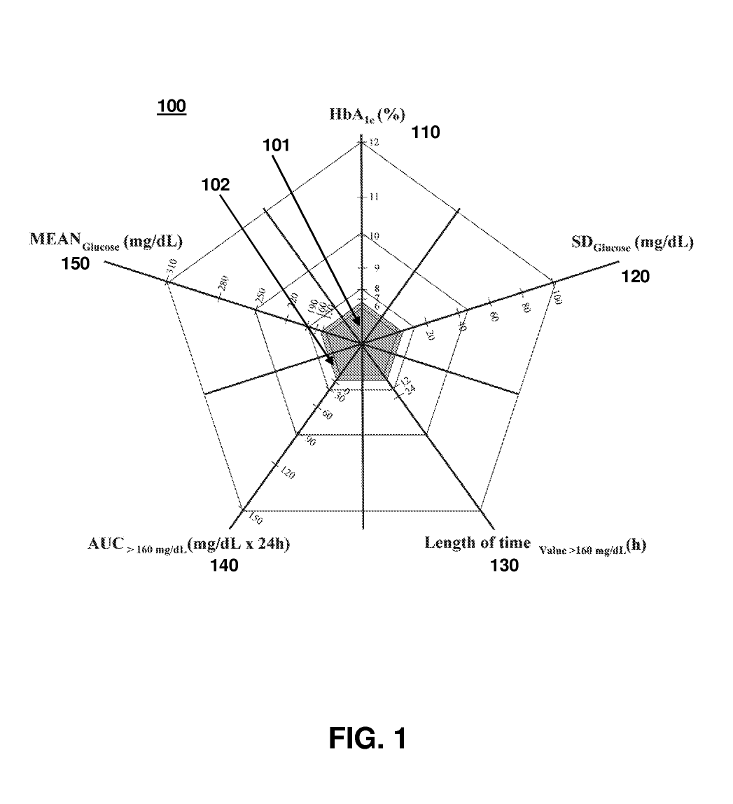

FIG. 1 illustrates a Glucose Pentagon according to embodiments of the present invention. Five parameters are calculated from glucose profiles of diabetic and non-diabetic (healthy) patients, and each parameter forms a single axis of a five-sided figure, the Glucose Pentagon 100:

the HbA_{1c} value (this is not calculated, but is instead incorporated as an existing value) axis 110
the standard deviation of the mean glucose concentration axis 120
the amount of time per day in hyperglycemic values (e.g., >160 mg/dL=8.9 mmol/L) axis 130
the area-under-curve (AUC) of hyperglycemic values (e.g., >160 mg/dL=8.9 mmol/L) axis 140
the mean glucose concentration axis 150.

Taken together, the selected parameters provide an integrated description of glycemia over the period of time under observation. These parameters also make it possible to incorporate other data indirectly, such as, for example, preprandial glycemia, postprandial glycemic excursions, and MAGE, whereby the mean glucose concentration describes the average glycemic situation and the standard deviation describes glycemic variability to a certain degree.

Using MAGE as a parameter for the Glucose Pentagon 100 instead of the standard deviation also may be an option, as this better characterizes extreme glycemic excursions. Measuring oxidative stress by determining the rate at which 8-iso PGF_{2α} is excreted in the urine yields a MAGE value of 45 mg/dL in individuals with healthy metabolism. Values up to approximately 150 mg/dL (mean: 75 mg/dL) have been recorded in patients with Type 2 diabetes; this value may range up to approximately 280 mg/dL (mean: 140 mg/dL) in patients with Type 1 diabetes. No definitive correlation has been demonstrated between this marker and MAGE (r=−0.381). Hence, according to embodiments of the present invention, MAGE was not taken into consideration in the Glucose Pentagon 100 of patients with Type 1 diabetes, although it may be still utilized in alternative embodiments of the present invention.

Including the HbA_{1c} value in the Glucose Pentagon 100 links the parameters determined from glucose profiles with what is recognized as the best parameter for characterizing long-term metabolic control. It is true that, to a large extent, a linear correlation (r=0.876) between the HbA_{1c} value and the mean glycemic value determined from continuous glucose monitoring (CGM) entries does exist. This correlation may be defined, for example, by the following equation:

mean glucose_{(CGM over 3 months)} [mmol/L]=1.649×HbA_{1c}−2.645

As such, this value is theoretically already represented in the Glucose Pentagon 100. If the information provided by the mean glucose concentration is to be as meaningful as that yielded by the HbA_{1c} value, however, the glucose profile must not contain any relatively long gaps over the 3-month time period under consideration. This issue has almost always been the case with day-to-day monitoring, however, at least up to now, which is why the HbA_{1c} value was incorporated into the Glucose Pentagon 100 according to embodiments of the present invention. Another advantage of integrating the HbA_{1c} value is that it provides a link to a verified laboratory diagnostic value covering a glycemic period of 8-10 weeks.

The time per day values and area-under-curve (AUC) per day values at blood glucose levels of, for example but not necessarily limited to, greater than 160 mg/dL are both parameters that characterize hyperglycemic phases over the course of a day, and are considered to be additional risk parameters for developing diabetic complications. The daily time AUC value clearly correlates with oxidative stress and, as such, is relevant to the development of vascular complications. These parameters are assigned their own independent significance, as both are only partially reflected in the calculated mean/standard deviation and the HbA_{1c} value. A value of 160 mg/dL is taken, for example, as the threshold value for normal glycemia and thus increased risk. This value was selected because it represents a typical, postprandial maximum value for individuals with healthy metabolism whose glucose profiles are recorded with CGM, although according to alternative embodiments of the present invention, any other suitable value may be utilized.

According to embodiments of the present invention, time and AUC in the hypoglycemic range are not taken into consideration directly, however, as these values do not correlate directly with the risk of developing diabetic complications. The controversial influence of hypoglycemic events on mortality rate may be surmised and should be primarily interpreted as an acute event in patients with existing vascular damage. Due to subsequent autonomic counter-regulation, however, hypoglycemic events do have an indirect impact on glycemic variability. This effect is encompassed by the standard deviation of the mean glucose concentration. In principle, rates of hypoglycemia, time of hypoglycemic events and AUC represent a trio of parameters lying outside the Glucose Pentagon 100.

The values taken into consideration here cover a surface area that is easy to calculate and that may be viewed as an independent, integrated parameter for describing glycemia. A meaningful way of obtaining a dimensionless value is to normalize this area using the following values recorded in CGM profiles of individuals with healthy metabolism. The resulting area is shown as inner pentagon 101 in FIG. 1. The values forming the inner pentagon 101 are as follows:
HbA_{1c} value: ≦5.5%
standard deviation of the glucose concentration: ±10 mg/dL (0.55 mmol/L)
time per day >160 mg/dL (8.9 mmol/L): 0 min
AUC >160 mg/dL (8.9 mmol/L): 0 mg/dL×day
mean glucose concentration: 90 mg/dL (5 mmol/L)

The area calculated for the glucose pentagon of a patient with diabetes, divided by the reference/standard pentagon area 101 of healthy individuals, provides a more meaningful assessment of a patient's risk of developing diabetic complications than is possible with just the HbA_{1c} value. The reason for this conclusion is that the Glucose Pentagon 100 incorporates parameters providing information on glycemic variability. This feature is not the case with HbA_{1c} alone.

The starting point for the axes 110, 120, 130, 140, 150 are determined using values from healthy individuals, whereby even in these cases values lie above zero. The influence of individual parameters on the risk of developing microvascular and macrovascular complications must be taken into consideration when selecting the scale of the axes. With respect to the HbA_{1c} value, studies have established this influence for patients with Type 1 and Type 2 diabetes. Risk curves for developing complications do not indicate the same degree of risk for microalbuminuria, neuropathy, nephropathy and retinopathy (Type 1 diabetes) and/or for microvascular or macrovascular end points (Type 2 diabetes). As such, a reasonable approach would be to define an average function that is based on these curves and dependent on the HbA_{1c} value. Theoretically, however, the Glucose Pentagon 100 also may be calculated specifically for each individual complication.

The mean glucose concentration is closely correlated to the HbA_{1c} value. The scale for the daily time AUC value in the hyperglycemic range, in turn, is oriented toward this mean glucose value, in that the threshold for hyperglycemia (160 mg/dL=8.9 mmol/L) is subtracted from each mean glucose value. Establishing the scale for the two other parameters is more difficult. No clinical study data on time spent in the hyperglycemic range is currently available. Reference therefore only may be made to studies on the rate of apoptosis in human umbilical endothelial cells under conditions of continuous and variable glycemia, whereby the relationship is presumably linear. We have likewise assumed a linear scale for the standard deviation value of the mean glucose concentration—an assumption based on various studies on the relationship between oxidative stress markers and glucose variability.

Of critical concern is the ability to estimate which errors will arise in the overall Glucose Pentagon 100 when individual parameters vary. Unlike the HbA_{1c} value, which yields virtually no information on glycemic variability when taken alone, the area of the Glucose Pentagon 100 provides a more extensive and better description. Because the HbA_{1c} value is entered into the model as a constant that does not change until the next measurement is taken, an “error” arises when this parameter briefly improves or worsens relative to its baseline. The maximum error caused by such a situation may be, however, estimated using the correlation between the mean glucose value and the HbA_{1c} value. The estimated error may be indicated for the HbA_{1c} value in the Glucose Pentagon 100 at any given point in time as ΔF_{HbA1c}, which represents the deviation of the current (but not of the most recently measured) HbA_{1c} value. This fact is immediately apparent in the Glucose Pentagon 100: the line connecting the axes for HbA_{1c} 110 and mean glucose 150 runs parallel to the edge of the standard pentagon area 101 if the mean glucose corresponds to the HbA_{1c} value. If the mean glucose value is “better” than the HbA_{1c} value, then the connecting line will be angled toward the center of the Glucose Pentagon 100 at the point where it meets the MEAN_{Glucose} axis 150; if the value is worse, the line will angle outwards.

Measurement errors that occur during the process of recording the glucose profile, or during the process of determining the HbA_{1c} value, also give rise to discrepancies between the mean glucose concentration and the HbA_{1c} value. “Errors” in HbA_{1c} measurements also may have pathological sources. Hemoglobinopathies or hemolytic anemia, for instance, yield false low values, whereas chronic iron deficiency anemia causes false high values for HbA_{1c}. Discrepancies of this type are immediately apparent in the Glucose Pentagon 100. This issue also may be confirmed by dividing the mean glucose concentration by the HbA_{1c} value—a concept similar to the Glyc-Q parameter, a value which is obtained through the division of fructosamine by HbA_{1c} (Glyc-Q=Fructosamine×2.2/HbA_{1c}).

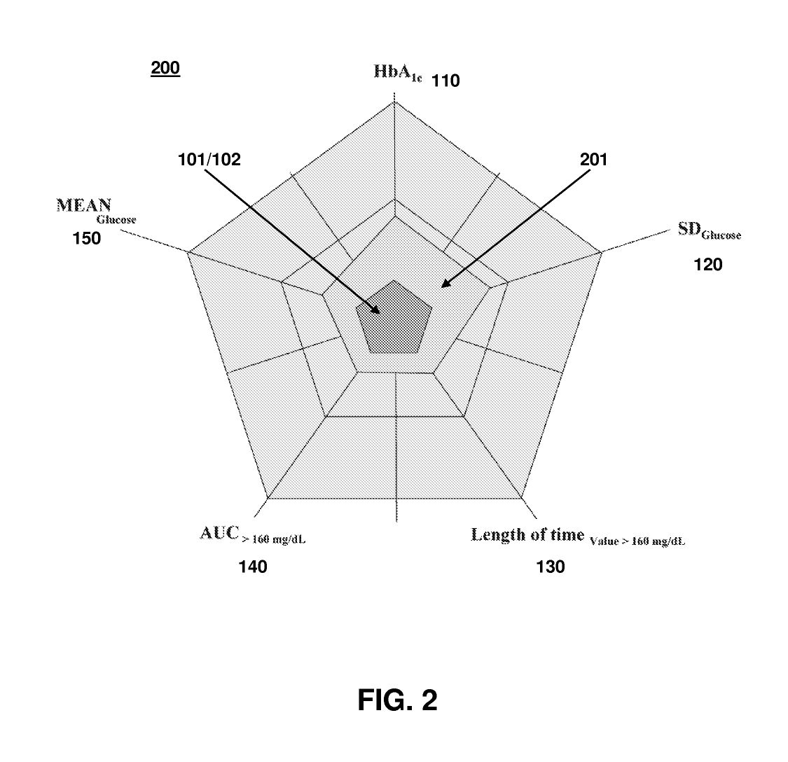

FIG. 2 illustrates a Glucose Pentagon comparing a pentagon of a non-diabetic person to a pentagon of a diabetic patient according to embodiments of the present invention. Taking the area of the glucose pentagon 201 for a diabetic patient, plotted utilizing five points on the five axes 110, 120, 130, 140, 150, respectively, indicative of the diabetic patient's HbA_{1c} and CGM values, and normalizing it to the standard area of the glucose pentagon 101 for a healthy/non-diabetic individual, plotted utilizing five points on the five axes 110, 120, 130, 140, 150, respectively, indicative of a healthy/non-diabetic person's HbA_{1c} and CGM values (or alternatively, normalizing it to the standard area of the glucose pentagon 102, as illustrated in FIGS. 1 and 2, representing the range in which the risk of diabetes patients developing diabetic complications is low, according to embodiments of the present invention) yields a non-dimensional characteristic value defined as the Glycemic Control Parameter or Glycemic Risk Parameter (GRP):

$$GRP = \frac{area of the glucose pentagon of a diabetic patient}{\begin{matrix}area of the glucose pentagon for healthy \text{/} \\ non \text{-} diabetic individual\end{matrix}}$$

This parameter quickly allows an assessment of a patient's metabolic control while taking significantly more factors into consideration than is possible by looking solely at the HbA_{1c} value. The GRP may be established as a relatively easily determined parameter that better describes an individual patient's risk of developing diabetic complications. A scale that offers a rapid overview of metabolic conditions on each individual day then may be developed in parallel. When viewed in their entirety over time, numerous values such as these will yield information comparable to the HbA_{1c} value because they incorporate data on acute and long-term glycemia; however such values will provide a significantly more comprehensive picture of the situation. Unlike the HbA_{1c} value, however, the GRP for a given time period is available at any time. Furthermore, the parameters integrated within the GRP also may be considered separately when a more detailed glycemic assessment is required.

Metabolic control may be subsequently assessed on two fundamental levels:
the GRP as an integrated parameter for assessing glycemia and as a parameter for monitoring daily success (risk control)
the individual parameters of mean, standard deviation, AUC, hyperglycemic time, and HbA_{1c} for a detailed glycemic assessment.

In practice, according to embodiments of the present invention, a suitable software system may be utilized as a simple means of determining the GRP and the individual parameters of the Glucose Pentagon 100 from a measured CGM profile. Such a system would not only calculate the values of the various parameters, but would also plot the corresponding chart and determine the GRP. Software integrated into CGM systems may be reprogrammed accordingly. The only required input is the most up-to-date HbA_{1c} value available.

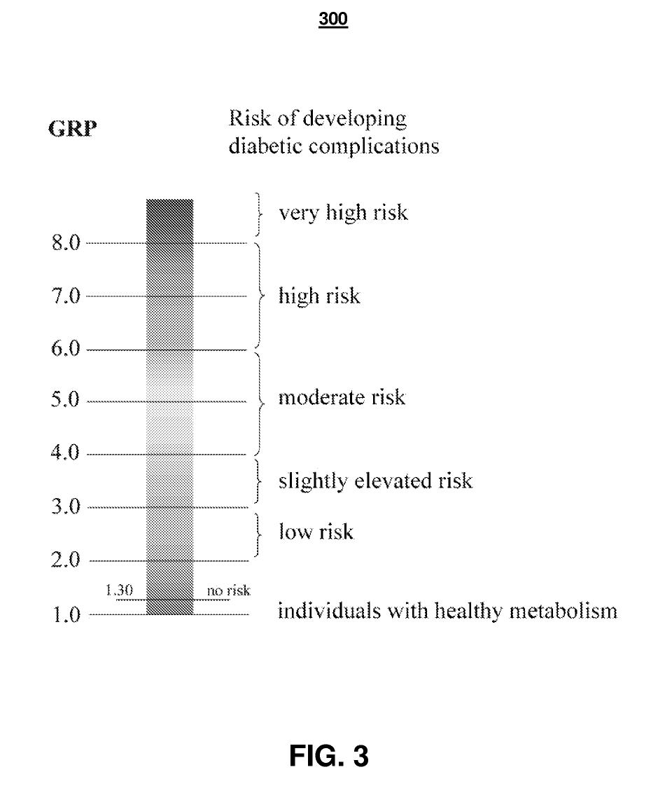

FIG. 3 illustrates a representative relationship between a Glycemic Risk Parameter (GRP) value and the risk of developing diabetic complications according to embodiments of the present invention. A graphic representation 300 of the calculated GRP, which may be color-coded according to the risk of developing diabetic complications according to embodiments of the present invention, may allow health professionals and patients to directly gauge the success of their efforts in order to optimize metabolic control. At the same time, concrete data and the other parameters underlying the Glucose Pentagon 100 may be available to the therapist for a more detailed analysis.

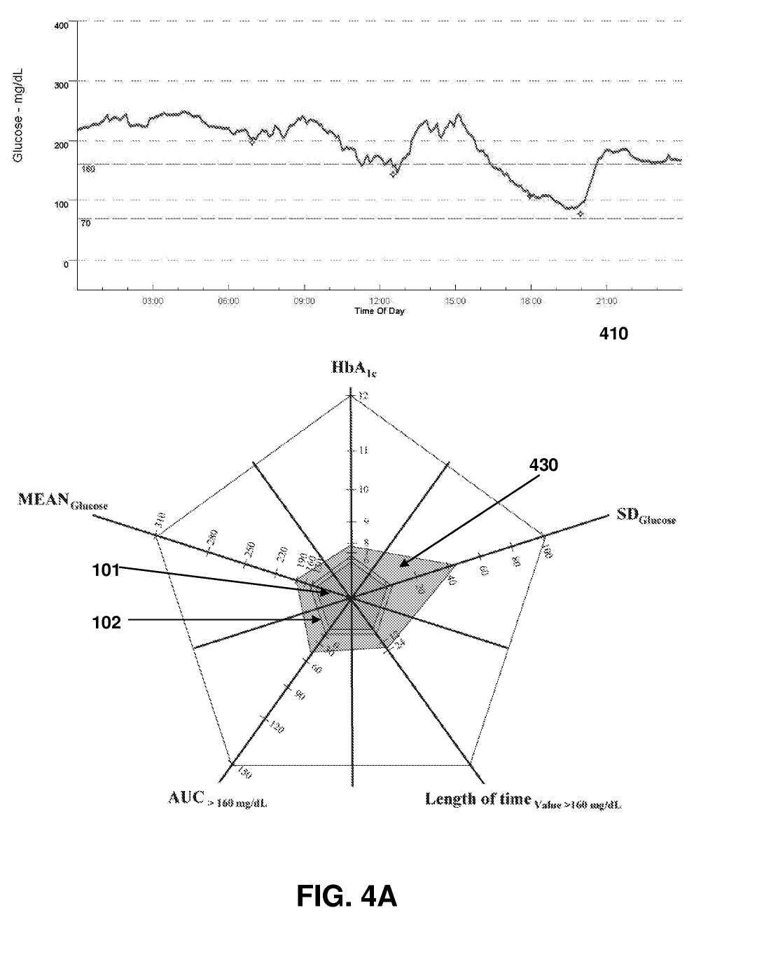

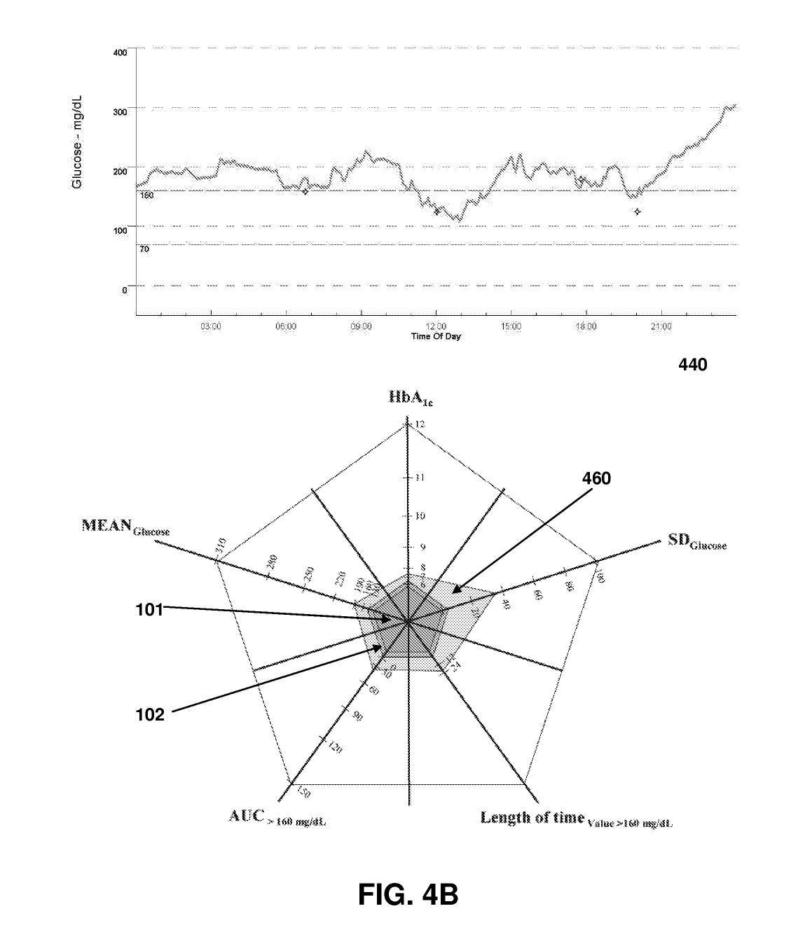

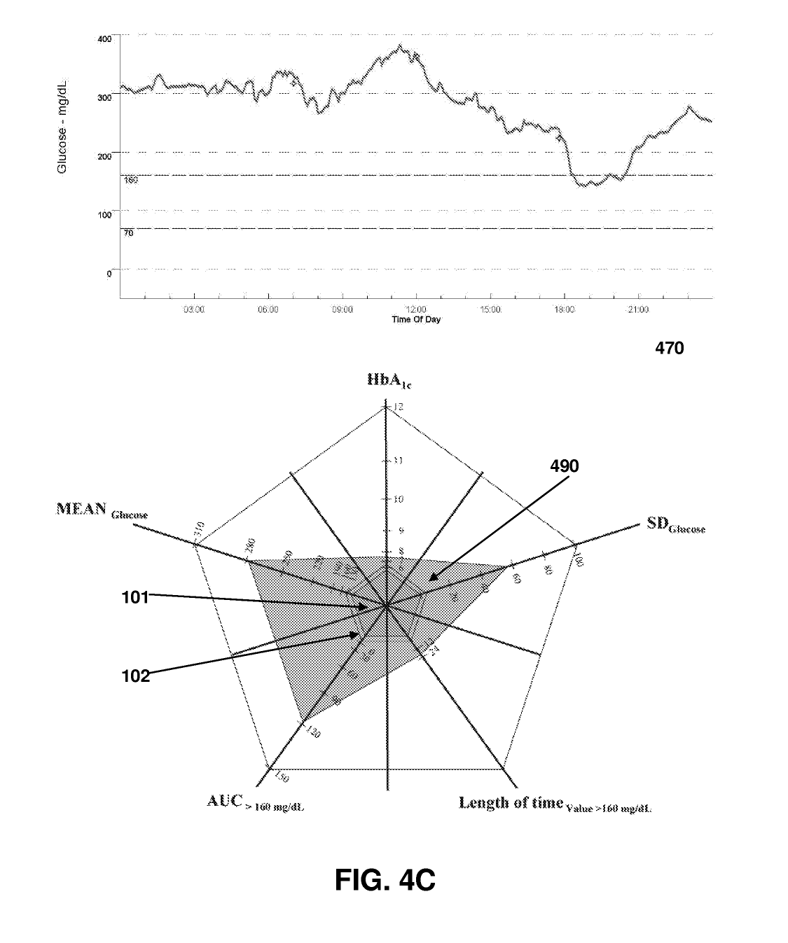

FIGS. 4A-4C illustrate sample continuous glucose monitoring (CGM) profiles over three days and the resultant Glucose Pentagons for a representative diabetic patient according to embodiments of the present invention. The following examples according to embodiments of the present invention use data from patients with Type 1 diabetes and are intended to illustrate how the Glucose Pentagon 100 may be used in practice. The patient's CGM data is represented in graphs 410, 440, 470 for Day 1, Day 2, and Day 3, respectively. An HbA_{1c} value of 7.5% had most recently been measured for a 49-year-old female patient with Type 1 diabetes who had suffered from diabetes for 40 years and managed her condition with insulin pump therapy combined with a rapid-acting analog insulin. Blood pressure and lipid parameters were well regulated with a beta-blocker, an ACE inhibitor, and a statin. Known conditions included retinopathy, nephropathy, peripheral neuropathy and stage 2 peripheral arterial occlusive disease (PAOD).

The underlying parameters and the resulting GRP are given in the following table (referring to FIGS. 4A, 4B, and 4C corresponding to Day 1, Day 2, and Day 3, respectively):

| | Day 1 | Day 2 | Day 3 |
|---|---|---|---|
| HbA_{1c} (%) | 7.5 | 7.5 | 7.5 |
| MEAN_{glucose} (mg/dL) | 191 | 188 | 278 |
| SD_{glucose} (mg/dL) | 44 | 34 | 57 |
| AUC_{>160 mg/dL} (mg/dL × day) | 39 | 32 | 118 |
| Time/day_{>160 mg/dL} (min.) | 1155 | 1230 | 1325 |
| GRP | 3.30 | 2.87 | 7.38 |

The average GRP from these three days, calculated from the glucose pentagons 430, 460, 490, relative to the reference non-diabetic/healthy glucose pentagon 101 (or 102), as illustrated in FIGS. 4A-4C, is 4.52, which indicates an increased risk of diabetic complications (referring to FIG. 3 according to embodiments of the present invention). One suspects that these values are typical for the patient, as clearly evidenced by the existing diabetic complications. The pattern in the glucose pentagons 430, 460, 490 also shows excursions toward high glucose variability on all days (standard deviation of the mean glucose concentration). Another noticeable characteristic is that the mean glucose concentration is higher than the current, most recently measured HbA_{1c} value for all three days. If these values represent the trend over a relatively long period of time, one might anticipate that the subsequent HbA_{1c} value will have worsened.

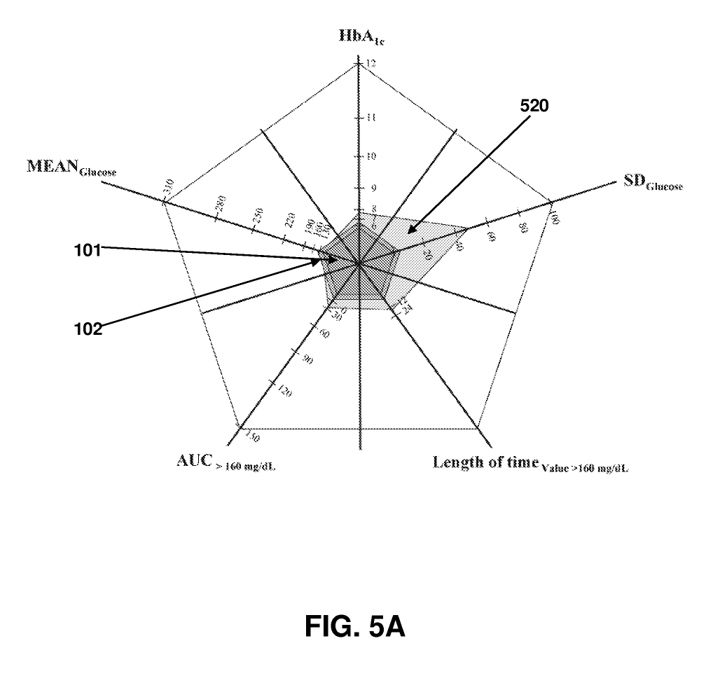

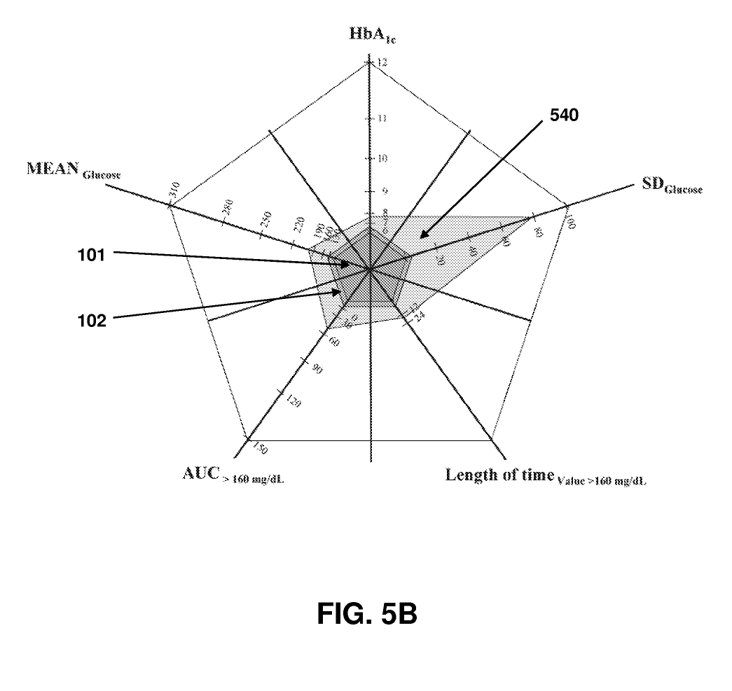

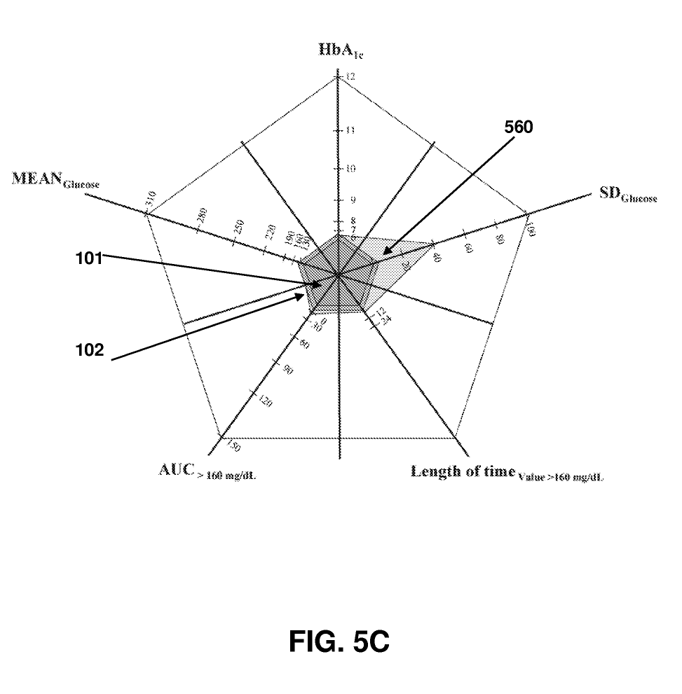

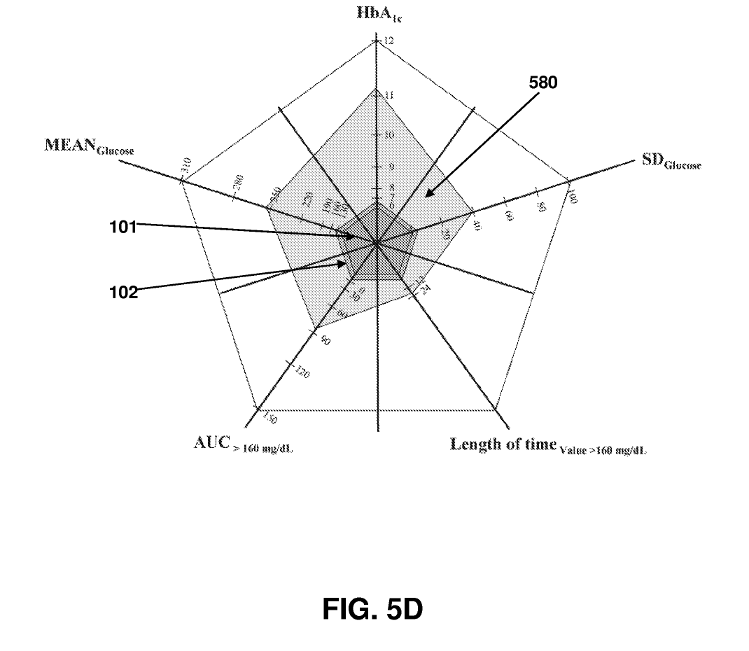

FIGS. 5A-5D illustrate Glucose Pentagons for a selected day for a plurality of diabetic patients. Referring to Table 1 below, CGM data is collected for three days for three representative diabetic patients, and their resulting GRP values and average three-day GRP values are calculated using the Glucose Pentagon 100 according to embodiments of the present invention. Patient JE includes data with analog insulin (a), and normal insulin (b), as indicated in Table 1 below.

TABLE 1

| | | | | | Avg. GRP |
|---|---|---|---|---|---|
| Patient | Data for calculations | Day 1 | Day 2 | Day 3 | (3 days) |
| JE, male, T1D, CSII | HbA_{1c} (%) | 7.7 | | | |
| Age: 40 | MEAN_{glucose} (mg/dL) | 115 | 111 | 128 | |
| Years with diabetes: 10, | SD_{glucose} (mg/dL) | 65 | 47 | 49 | |
| no complications | AUC_{>160 mg/dL} (mg/dL × | 14 | 25 | 9 | |
| a) with analog insulin | day) | | | | |
| b) with normal insulin | Time/day_{>160 mg/dL} (min.) | 305 | 865 | 270 | |
| | GRP | 2.99 | 2.82 | 2.62 | 2.81 |
| | HbA_{1c} (%) | 7.7 | | | |
| | MEAN_{glucose} (mg/dL) | 153 | 200 | 175 | |
| | SD_{glucose} (mg/dL) | 59 | 79 | 62 | |
| | AUC_{>160 mg/dL} (mg/dL × | 22 | 52 | 33 | |
| | day) | | | | |
| | Time/day_{>160 mg/dL} (min.) | 630 | 980 | 870 | |
| | GRP | 3.07 | 4.22 | 3.52 | 3.60 |
| HB, male, T1D, ICT | HbA_{1c} (%) | 6.2 | | | |
| Age: 60 | MEAN_{glucose} (mg/dL) | 124 | 119 | 164 | |
| Years with diabetes: 36, | SD_{glucose} (mg/dL) | 35 | 41 | 37 | |
| complications: | AUC_{>160 mg/dL} (mg/dL × | 2 | 4 | 17 | |
| retinopathy, | day) | | | | |
| nephropathy, neuropathy | Time/day_{>160 mg/dL} (min.) | 315 | 255 | 770 | |
| Analog insulins | GRP | 2.10 | 2.03 | 2.20 | 2.11 |
| CT, female, T1D, ICT | HbA_{1c} (%) | 11.3 | | | |
| Age: 17 | MEAN_{glucose} (mg/dL) | 274 | 255 | 262 | |
| Years with diabetes: 4, | SD_{glucose} (mg/dL) | 55 | 38 | 70 | |
| complications: | AUC_{>160 mg/dL} (mg/dL × | 94 | 81 | 75 | |
| neuropathy | day) | | | | |
| Normal insulin/NPH | Time/day_{>160 mg/dL} (min.) | 1340 | 1250 | 1280 | |
| insulin | GRP | 10.59 | 8.41 | 9.97 | 9.66 |

FIGS. 5A-5D illustrate the corresponding glucose pentagons on one day selected for each patient in Table 1 above. The glucose pentagons 520, 540, 560 for the first two patients (JE in FIGS. 5A and 5B, and HB in FIG. 5C) are characterized predominantly by glycemic variability, whereas the high HbA_{1c} value and mean glucose concentration are responsible for the large pentagon 580 for patient CT in FIG. 5D. The comparison between the use of normal insulin (FIG. 5B) and rapid-acting analog insulin (FIG. 5A) in patient JE shows reduced glycemic variability with the analog insulin, resulting in better metabolic control. This kind of analysis is not possible when taking only the HbA_{1c} value into consideration and demonstrates the sense in combining long-term and acute glycemia within the GRP parameter according to embodiments of the present invention.

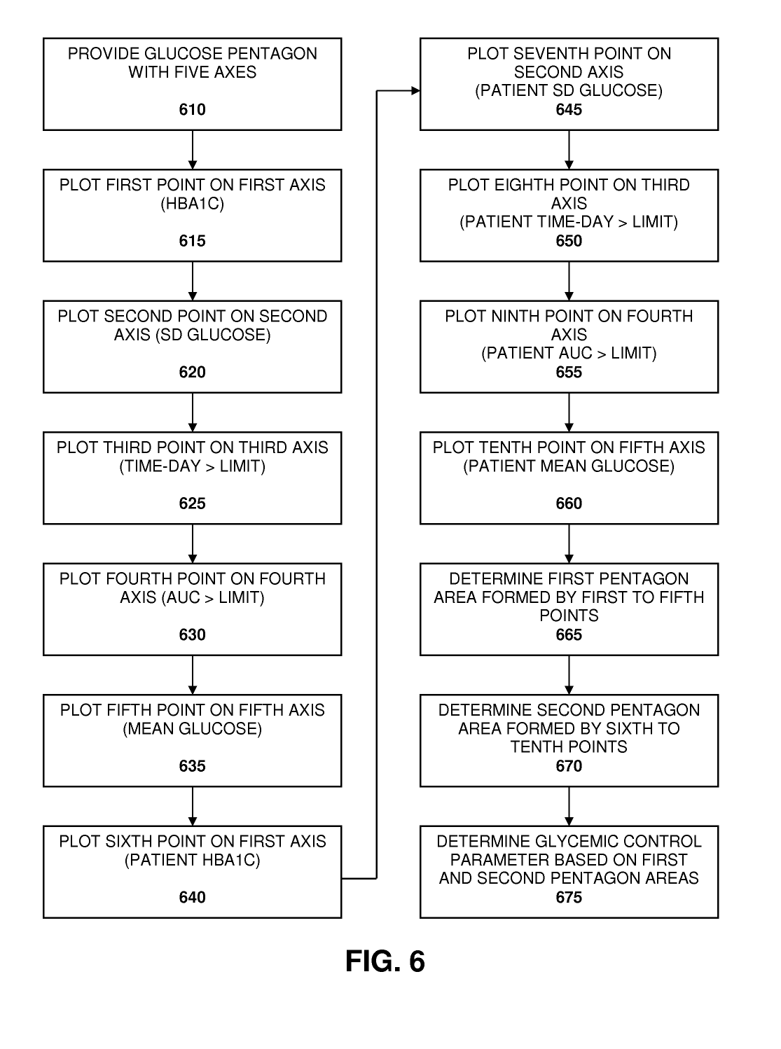

FIG. 6 illustrates a flow chart of evaluating glycemic control of a patient according to embodiments of the present invention. At step 610, a pentagon having five axes radiating from a center of the pentagon (see, e.g., Glucose Pentagon 100, FIG. 1) is provided. A first axis 110 (FIG. 1) has a length representing a range of hemoglobin A_{1c} values. A second axis 120 (FIG. 1) has a length representing a range of standard deviation of glucose values. A third axis 130 (FIG. 1) has a length representing a range of amount of time per day values exceeding a first limit. A fourth axis 140 (FIG. 1) has a length representing a range of daily area-under-curve values exceeding a second limit. A fifth axis 150 (FIG. 1) has a length representing a range of mean glucose values. According to embodiments of the present invention, the first axis representing the range of hemoglobin A_{1c} values and the fifth axis representing the range of mean glucose values may be adjacent to each other in the pentagon.

At step 615, a first point on the first axis 110 (FIG. 1) indicative of a representative hemoglobin A_{1c} value is plotted. At step 620, a second point on the second axis 120 (FIG. 1) indicative of a representative standard deviation of glucose value is plotted. At step 625, a third point on the third axis 130 (FIG. 1) indicative of a representative amount of time per day value exceeding the first limit is plotted. According to embodiments of the present invention, the first limit may be 160 mg/dL. At step 630, a fourth point on the fourth axis 140 (FIG. 1) indicative of a representative daily area-under-curve value exceeding a second limit is plotted. According to embodiments of the present invention, the second limit may be 160 mg/dL. At step 635, a fifth point on the fifth axis 150 (FIG. 1) indicative of a representative mean glucose value is plotted.

At step 640, a sixth point on the first axis 110 (FIG. 1) indicative of a hemoglobin A_{1c} value of the patient is plotted. At step 645, a seventh point on the second axis 120 (FIG. 1) indicative of a standard deviation of glucose value of the patient is plotted. At step 650, an eight point on the third axis 130 (FIG. 1) indicative of an amount of time per day value exceeding the first limit of the patient is plotted. At step 655, a ninth point on the fourth axis 140 (FIG. 1) indicative of a daily area-under-curve value exceeding the second limit of the patient is plotted. At step 660, a tenth point on the fifth axis 150 (FIG. 1) indicative of a mean glucose value of the patient is plotted.

A first pentagon area (see, e.g., pentagon 101 in FIG. 2) formed by the first point, the second point, the third point, the fourth point, and the fifth point on axes 110, 120, 130, 140, 150, respectively, is determined at step 665. A second pentagon area (see, e.g., pentagon 201 in FIG. 2) formed by the sixth point, the seventh point, the eighth point, the ninth point, and the tenth point on axes 110, 120, 130, 140, 150, respectively, is determined at step 670. At step 675, a glycemic control parameter (or Glycemic Risk Parameter—GRP) is determined based on the first pentagon area 101 and the second pentagon area 201. According to embodiments of the present invention, the glycemic control parameter (or Glycemic Risk Parameter—GRP) is determined by dividing the second pentagon area 201 (FIG. 2) by the first pentagon area 101 (FIG. 1).

According to embodiments of the present invention, the representative hemoglobin A_{1c} value, the representative standard deviation of glucose value, the representative amount of time per day value exceeding the first limit, the representative daily area-under-curve value exceeding the second limit, and the representative mean glucose value plotted to determine the first pentagon area 101 (FIGS. 1 and 2) are representative of a non-diabetic/healthy individual.

The evaluation of glycemic control of a patient according to embodiments of the present invention may be implemented on a computing device such as a computer system (e.g., desktop, laptop, enterprise systems, network/Web systems, etc.), a handheld device (e.g., PDAs), a mobile/smart phone, a medical device, an infusion device (e.g., insulin pumps), an infusion device controller/programmer, a hospital monitor, or any other suitable electronic device. Moreover, an article of manufacture (such as, e.g., a memory storage device such as a RAM/ROM, optical disk, flash memory, hard disk drive, etc., a computing device such as a computer system (e.g., desktop, laptop, enterprise systems, network/Web systems, etc.), a handheld device (e.g., PDAs), a mobile/smart phone, a medical device, an infusion device (e.g., insulin pumps), an infusion device controller/programmer, a hospital monitor, or any other suitable electronic device) containing code for evaluating glycemic control of a patient as discussed above, comprising a computer-usable medium including at least one embedded computer program that is capable of causing at least one computer to perform the evaluation of glycemic control of a patient as discussed above according to embodiments of the present invention, also may be utilized.

The Glucose Pentagon 100 provides an integrated description of glycemia in diabetic patients over a specific time interval, while it also includes independent factors for assessing metabolic control. The time interval may be even just a single day, according to embodiments of the present invention, which is a useful feature. The HbA_{1c} value is the only parameter that remains constant until it is measured again, but because it typically changes very little during short time intervals, the resulting error may be assumed to be negligible. This error does increase, however, the older the HbA_{1c} measurement is and the more the value changes. The Glucose Pentagon 100 is much less subject to error, however, than an assessment of metabolic control based solely on the HbA_{1c} value.

According to embodiments of the present invention, it would presumably make sense to determine the GRP for each individual day according to embodiments of the present invention so that the patient may use the pentagon to assess their day-to-day efforts. A mean GRP value then may be calculated over longer periods of time.

One advantage of embodiments of the present invention is that it takes both long-term and acute metabolic control into account, i.e., it unites HbA_{1c} and glycemic fluctuations in a single model. Because it yields a characteristic numerical value, the GRP serves as a good starting point for assessing the risk of developing diabetic complications and provides far more information than the HbA_{1c} value on its own. Detailed insight may be derived from the shape of the pentagon, which provides a quick overview of a patient's daily routine without having to look at the statistical details of the CGM profile. It also serves as a reference point for long-term care and clinical research. For the model to be useful, according to embodiments of the present invention, CGM software may perform glucose pentagon calculations and provide an opportunity for entering the HbA_{1c} value.

Specialized glucose pentagons also may be determined according to embodiments of the present invention in addition to the integrated glucose pentagon. These embodiments likewise may be incorporated into the CGM software, providing information on various types of diabetic complications, such as retinopathy, neuropathy, nephropathy, and cardiovascular events. It may be helpful to also distinguish between glucose pentagons for patients with Type 1 diabetes and those for individuals with Type 2 diabetes.

The inclusion of additional parameters, such as MAGE or preprandial glucose, is also conceivable according to embodiments of the present invention, as these independent parameters likewise represent risk factors for developing microvascular and macrovascular complications. In this case, according to embodiments of the present invention, the MAGE value may replace the standard deviation of the mean glucose concentration value. The addition of further parameters, such as patient age, and years with diabetes, etc., is also conceivable according to embodiments of the present invention. The basic model for calculating the area encompassed by the parameters and for normalizing this area against the pentagon for individuals with normal metabolism would remain the same. The Glucose Pentagon 100 may then become a “Glucose Polygon” according to embodiments of the present invention.

While the description above refers to particular embodiments of the present invention, it will be understood that many modifications may be made without departing from the spirit thereof. The accompanying claims are intended to cover such modifications as would fall within the true scope and spirit of the present invention.

The presently disclosed embodiments are therefore to be considered in all respects as illustrative and not restrictive, the scope of the invention being indicated by the appended claims, rather than the foregoing description, and all changes which come within the meaning and range of equivalency of the claims are therefore intended to be embraced therein.

1. A method of evaluating glycemic control of a patient, comprising:
providing a pentagon having five axes radiating from a center of the pentagon, wherein
a first axis has a length representing a range of hemoglobin A_{1c} values,
a second axis has a length representing a range of standard deviation of glucose values,
a third axis has a length representing a range of amount of time per day values exceeding a first limit;
a fourth axis has a length representing a range of daily area-under-curve values exceeding a second limit, and
a fifth axis has a length representing a range of mean glucose values;
plotting a first point on the first axis indicative of a representative hemoglobin A_{1c} value;
plotting a second point on the second axis indicative of a representative standard deviation of glucose value;
plotting a third point on the third axis indicative of a representative amount of time per day value exceeding the first limit;
plotting a fourth point on the fourth axis indicative of a representative daily area-under-curve value exceeding a second limit;
plotting a fifth point on the fifth axis indicative of a representative mean glucose value;
plotting a sixth point on the first axis indicative of a hemoglobin A_{1c} value of the patient;
plotting a seventh point on the second axis indicative of a standard deviation of glucose value of the patient;
plotting an eight point on the third axis indicative of an amount of time per day value exceeding the first limit of the patient;
plotting a ninth point on the fourth axis indicative of a daily area-under-curve value exceeding the second limit of the patient;
plotting a tenth point on the fifth axis indicative of a mean glucose value of the patient;
determining a first pentagon area formed by the first point, the second point, the third point, the fourth point, and the fifth point;
determining a second pentagon area formed by the sixth point, the seventh point, the eighth point, the ninth point, and the tenth point; and
determining a glycemic control parameter based on the first pentagon area and the second pentagon area.

2. The method of claim 1, wherein the glycemic control parameter is determined by dividing the second pentagon area by the first pentagon area.

3. The method of claim 1, wherein the representative hemoglobin A_{1c} value, the representative standard deviation of glucose value, the representative amount of time per day value exceeding the first limit, the representative daily area-under-curve value exceeding the second limit, and the representative mean glucose value are representative of a non-diabetic individual.

4. The method of claim 1, wherein the first limit is 160 mg/dL.

5. The method of claim 1, wherein the second limit is 160 mg/dL.

6. The method of claim 1, wherein the method is implemented on a computing device.

7. The method of claim 1, wherein the method is implemented on an infusion device.

8. The method of claim 1, wherein the method is implemented on an infusion device controller/programmer.

9. The method of claim 1, wherein the method is implemented on a medical device.

10. The method of claim 1, wherein the first axis representing the range of hemoglobin A_{1c} values and the fifth axis representing the range of mean glucose values are adjacent to each other in the pentagon.

11. An article of manufacture containing code for evaluating glycemic control of a patient, comprising a computer-usable medium including at least one embedded computer program that is capable of causing at least one computer to perform:
providing a pentagon having five axes radiating from a center of the pentagon, wherein
a first axis has a length representing a range of hemoglobin A_{1c} values,
a second axis has a length representing a range of standard deviation of glucose values,
a third axis has a length representing a range of amount of time per day values exceeding a first limit;
a fourth axis has a length representing a range of daily area-under-curve values exceeding a second limit, and
a fifth axis has a length representing a range of mean glucose values;
plotting a first point on the first axis indicative of a representative hemoglobin A_{1c} value;
plotting a second point on the second axis indicative of a representative standard deviation of glucose value;
plotting a third point on the third axis indicative of a representative amount of time per day value exceeding the first limit;
plotting a fourth point on the fourth axis indicative of a representative daily area-under-curve value exceeding a second limit;
plotting a fifth point on the fifth axis indicative of a representative mean glucose value;
plotting a sixth point on the first axis indicative of a hemoglobin A_{1c} value of the patient;
plotting a seventh point on the second axis indicative of a standard deviation of glucose value of the patient;
plotting an eight point on the third axis indicative of an amount of time per day value exceeding the first limit of the patient;
plotting a ninth point on the fourth axis indicative of a daily area-under-curve value exceeding the second limit of the patient;
plotting a tenth point on the fifth axis indicative of a mean glucose value of the patient;
determining a first pentagon area formed by the first point, the second point, the third point, the fourth point, and the fifth point;
determining a second pentagon area formed by the sixth point, the seventh point, the eighth point, the ninth point, and the tenth point; and
determining a glycemic control parameter based on the first pentagon area and the second pentagon area.

12. The article of claim 11, wherein the glycemic control parameter is determined by dividing the second pentagon area by the first pentagon area.

13. The article of claim 11, wherein the representative hemoglobin A_{1c} value, the representative standard deviation of glucose value, the representative amount of time per day value exceeding the first limit, the representative daily area-under-curve value exceeding the second limit, and the representative mean glucose value are representative of a non-diabetic individual.

14. The article of claim 11, wherein the first limit is 160 mg/dL.

15. The article of claim 11, wherein the second limit is 160 mg/dL.

16. The article of claim 11, wherein the article is a computing device.

17. The article of claim 11, wherein the article is an infusion device.

18. The article of claim 11, wherein the article is an infusion device controller/programmer.

19. The article of claim 11, wherein the article is a medical device.

20. The article of claim 11, wherein the first axis representing the range of hemoglobin A_{1c} values and the fifth axis representing the range of mean glucose values are adjacent to each other in the pentagon.
